(19) United States
(12) Patent Application Publication
(10) Pub. No.: US 2011/0256580 A1
(43) Pub. Date: Oct. 20, 2011

(54) LC-MFR-MS-Based Method and Apparatus for Screening a New Drug Candidate

(75) Inventors: Sanggoo Kim, Seoul (KR); Jin-Woo Jung, Seoul (KR); Chan-Soo Park, Seoul (KR)

(73) Assignee: KOREA BASIC SCIENCE INSTITUTE, Daejeon (KR)

(21) Appl. No.: 13/141,727

(22) PCT Filed: Dec. 16, 2009
(86) PCT No.: PCT/KR2009/007531

§ 371 Date: Jul. 1, 2011

(30) Foreign Application Priority Data
Dec. 26, 2008 (KR) 10-2008-0134719

(51) Int. Cl. G01N 21/75; C12Q 1/02; G01N 30/16; G01N 21/64

(52) U.S. Cl. 435/29; 436/172; 436/86; 436/94; 422/68.1; 422/70

(57) ABSTRACT

The present invention relates to a method for screening a new drug candidate using a liquid chromatography/microfluidic device/mass spectrometry system, and to a liquid chromatography/microfluidic device/mass spectrometry system. The present invention involves the detection of an interaction between molecules on a real-time basis through adjustment of a microreaction between traces of natural material or synthesized new drug candidates and a target material (protein or cell, etc.), thus developing materials for new drug candidates at a lower cost and with high efficiency, while improving quality of life and reducing medical costs. The present invention can be valuably used in increasing new scientific technology through the convergence of nanotechnology, biotechnology, and analytical chemistry technology.

### TECHNICAL FIELD

The present invention relates to a Liquid chromatography(LC)-microfluidic device(MFR)-mass spectrometry(MS)-based method and apparatus for screening a new drug candidate.

### BACKGROUND ART

New drug development generally consists of new drug material screening stage, preclinical stage, clinical stage (phase 1, phase 2, phase 3), and commercialization stage, and requires at least five years for the development stage and more than a billion dollars for the development and clinical tests. However, once the efficacy and safety are verified, this potential blockbuster drug can generate several trillion dollars of revenues (see FIG. 1). However, many new drug candidate materials do not pass the preclinical and clinical trial stages due to safety and toxicity problems. Accordingly, it is required to drop out the materials that have low possibility for commercialization or drugs with toxicity as early as possible to save cost and time for development of new drugs.

In order to resolve the above-mentioned problems, screening may generally be performed to select the new drug candidate materials. The screening method of new drug material generally involves in-vitro assay and cell-based assay, and it is generally necessary that low molecular compounds which act on the proteins that have an established correlativity to specific genes and diseases are developed. Accordingly, faster and more efficient screening techniques are necessary, which can ensure that the candidate materials for new drug development are obtained as many as possible.

Meanwhile, the focus of new drug development and research has recently extended from the synthesized to natural drugs. The natural drugs, which can be obtained from plants or marine microorganisms, are widely known as being free of side-effect even with a long period of use.

Particularly in South Korea where infrastructure for new drug development has not been built completely, the field of new drug development using natural materials has gained a place as the Blue Ocean strategy. The synthesized drugs can also use the natural extract as a starting material. Considering the fact that approximately 25% of new drug materials fail the toxicity test, the drugs using natural materials are expected to have higher possibility to be free of toxicity than synthetic counterpart. Given the above-mentioned situation, the natural drugs have been steadily developed for commercialization thereof, and the Korean government and science field have legislated related laws to promote and encourage natural drug development. Since the natural materials generally include a trace of candidate material with other compounds, it may be desirable that the screening incorporates a method for isolating and purifying only the reactant material, to thereby shorten time for development.

In order to discover new drug candidate from the natural extract, it is necessary to confirm interaction between the separated candidate with proteins related to disease-associated protein.

The interaction between biomaterial such as protein with separated candidate can generally be measured using bioluminescence resonance energy transfer (BRET), fluorescence resonance energy transfer (FRET), fluorescence polarization (FP), etc., which utilize the principle of energy transfer according to interaction between the materials. These methods can observe changes in cells or proteins with relatively high degree of speed and accuracy, and thus are used in the screening of new drug materials that can react with the same.

BRET uses luciferin, which is extracted from firefly, as a light-emitting resonator to detect interaction between molecules when the molecules approach, i.e., when the resonance energy between donor and acceptor materials is transferred.

FRET is capable of measuring and detecting the energy transferred to the acceptor molecule based on fluorescent energy transferred as the resonance energy from the interaction between molecules is transferred.

FP is particularly utilizable in the field of new drug substance screening, since FP can acquire physical information regarding molecular movement including protein-ligand binding, protein-nucleic aid interaction, protein degradation, or the like.

Like BRET or FRET, in FP, resonance energy, instead of energy by photon, is transferred to cause variation in fluorescence when the substances are in vicinity to each other. One of the advantages of FP is lower dependency on the fluorescent material than FRET, which allows test to be conducted under a variety of biological environments. Due to anisotropy, FP varies depending on rotation and mass of the molecules, and macromolecules such as protein exhibit high degree of fluorescence polarization during rotation. By utilizing the such characteristics, it is possible to measure interaction between the molecules. That is, it is possible to find that whether a material binds to the protein or not by measuring the interaction between ligand and protein with fluorescence or fluorescent polarization. Therefore, it is possible to selectively differentiate the new drug candidate substances using the above-explained assays.

Accordingly, the present inventors have designed a microfluidic device which can adjust micro-reaction between natural material containing a new drug candidate material or synthetic new drug with a target material, and detect interaction between molecules based on an increase of fluorescence, and developed and completed a screening method, according to which the new drug candidate separated with the liquid chromatograph is fed into the microfluidic device and a mass spectrometer, so that the microfluidic device carries out detection at multi levels using only a trace of substance, and also according to which analysis result is provided in real-time basis, and an apparatus for implementing said method.

### DISCLOSURE

### Technical Problem

An object of the present invention is to provide a method for screening a new drug candidate using liquid chromatography/microfluidic device/mass spectrometry system.

Another object of the present invention is to provide the microfluidic device.

Yet another object of the present invention is to provide a liquid chromatography/microfluidic device/mass spectrometry system to implement the method for screening the new drug candidate.

Further another object of the present invention is to provide a method for constructing a library of new drug candidate materials according to which a mixture of the new drug candidate materials is separated with liquid chromatograph and reacted.

Yet another object of the present invention is to provide a method for identifying a reaction between the new drug candidate material and a target material.

Further another object of the present invention is to provide an apparatus for identifying a reaction between the new drug candidate material and a target material.

### Technical Solution

In order to accomplish the above-mentioned object, the present invention provides a method for screening a new drug candidate material using a liquid chromatography/microfluidic device/mass spectrometry system.

Also, the present invention provides the microfluidic device.

Further, the present invention provides a liquid chromatography/microfluidic device/mass spectrometry system to implementing the method for screening.

Also, the present invention provides a method for constructing a library of new drug candidate materials according to which a mixture of the new drug candidate materials is separated with liquid chromatograph and reacted.

Further, the present invention provides a method for identifying a reaction between the new drug candidate material and a target material.

Also, the present invention provides an apparatus for identifying a reaction between the new drug candidate material and a target material.

### Advantageous Effects

The present invention involves the detection of an interaction between molecules on a real-time basis through adjustment of a micro-reaction between traces of natural material or synthesized new drug candidates and a target material, thus developing materials for new drug candidates at a lower cost and with higher efficiency, while improving quality of life and reducing medical costs. The present invention can also be useful in increasing new scientific technology through the convergence of nanotechnology, biotechnology, and analytical chemistry technology.

### BRIEF DESCRIPTION OF THE DRAWINGS

FIG. 1 is a graph related to drug development process and material value;

FIG. 2 is an image of microfluidic device connected to a liquid chromatograph according to an embodiment of the present invention;

FIG. 3 illustrates a microfluidic device (in Y-shaped connection) according to an embodiment of the present invention;

FIG. 4 illustrates a microfluidic device (in T-shaped connection) according to an embodiment of the present invention;

FIG. 5 is a conceptual view of a liquid chromatography/microfluidic device/mass spectrometry system according to an embodiment of the present invention;

FIG. 6 shows an actual image of a liquid chromatography/microfluidic device/mass spectrometry system according to an embodiment of the present invention, in which FIG. 6(a) shows a whole apparatus and FIG. 6(b) shows nano column connected to mass spectrometer;

FIG. 7 illustrates a microfluidic device (in Y-shaped connection) according to an embodiment of the present invention;

FIG. 8 is a conceptual view of a liquid chromatography/microfluidic device/mass spectrometry system with a splitter-integrated microfluidic device according to an embodiment of the present invention;

FIG. 9 shows a fluorescent image and a spreadsheet of a method for identifying a reactant according to an embodiment of the present invention, in which FIG. 9(a) shows fluorescent image of step 3 and FIG. 9(b) shows the spreadsheet of step 4;

FIG. 10 is an image showing droplets formed in the microfluidic device in order of A->B->C->D, according to an embodiment of the present invention;

FIG. 11 is an image showing droplets formed in the microfluidic device, in which FIG. 11(a) shows the droplets formed in order of A->B->C->D and FIG. 11(b) shows the droplets formed uniformly, according to an embodiment of the present invention;

FIG. 12 illustrates the microfluidic device mounted on the fluorescent microscope in Experimental example 1 according to an embodiment;

FIG. 13 is a microscopic image showing fluorescence of a new drug material screened in Experimental example 1, according to an embodiment of the present invention;

FIG. 14 is a microscopic image showing fluorescence of a new drug material screened in Experimental example 2, according to an embodiment of the present invention;

FIG. 15 is a mass spectrometry data of the new drug substance screened in Experimental example 2, according to an embodiment; and

FIG. 16 is a microscopic image showing fluorescence of a new drug material screened in Experimental example 3 fluoresces, according to an embodiment of the present invention.

### BEST MODE

The present invention will be explained in detail below.

The present invention provides a method for screening a new drug candidate material using a liquid chromatography/microfluidic device/mass spectrometry system, the method comprising: separating a natural material containing the new drug candidate material or a synthesized new drug material using a liquid chromatograph (step 1); injecting the new drug candidate material separated at step 1 into inlets of a mass spectrometer and of a microfluidic device at the same time through a splitter (step 2-1); injecting a target material into an inlet of a target material channel designed to be connected to a new drug candidate material channel for the new drug candidate material separated at step 1 (step 2-2); reacting the new drug candidate material and the target material injected at steps 2-1 and 2-2 respectively in a reaction channel connected with the channels of steps 2-1 and 2-2 (step 3); injecting an oil through an oil channel connected to the reaction channel of step 3 to form droplets (step 4); and detecting the droplets in fluorescence or emitting light from among the droplets of step 4, using the mass spectrometer (step 5).

The present invention will be explained in greater detail below with reference to the respective steps.

First, the step 1 according to present invention is a step separating natural material containing a new drug candidate material or a synthesized new drug candidate material using liquid chromatograph.

In particular, the step 1 according to present invention is a step separating a single new drug candidate material from the natural materials containing the new drug candidate materials or the synthesized new drug material using chromatography. Each new drug candidate material may be separated at different discharge time due to varying moving speed in columns.

Next, the step 2-1 according to the present invention is a step injecting the new drug candidate material separated at step 1 into the mass spectrometer and the new drug candidate material inlet of the microfluidic device through the splitter.

At step 2-1, the new drug candidate material may desirably be injected to the new drug candidate material inlet and the mass spectrometer of the microfluidic device at the same time, to find a new drug candidate material which is injected into the microfluidic device and reacted with the target material, and also to confirm the material injected into the mass spectrometer for analysis at the discharge time of the new drug candidate material separated at step 1.

Next, the step 2-2 according to the present invention is a step injecting the target material into the inlet of the target material channel which is designed to be connected to the new drug candidate material channel of step 2-1.

The new drug candidate material of step 2-1 or the target material of step 2-2 may preferably be injected in a flow rate of 1 to 20 μl/hour. If the flow rate is below 1 μl/hour, the new drug candidate material or the target material may not flow along the microchannel, while if the flow rate exceeds 20 μl/hour, the new drug candidate material or the target material may be accelerated too much to allow the reaction to occur between the two substances, according to which illumination or fluorescence sufficient to result in detection may not occur.

The target material of step 2-2 may preferably be a DNA, protein, cell or disease-associated material, and may be the one that can induce or increase fluorescence or illumination from a reaction with the new drug candidate.

Wherein, the target material may have a concentration from 0.1 to 10 mg/ml. If the concentration is less than 0.1 mg/ml, detection can be difficult.

It is also preferable that steps 2-1 and 2-2 are carried out concurrently so that appropriate reaction mixture can be provided to the reaction channel of step 3.

Next, step 3 according to the present invention is a step reacting the new drug candidate material and the target material injected at steps 2-1, 2-2 respectively with each other in a reaction channel which is connected to the new drug candidate material channel and the target material channel.

To be specific, the step 3 provides sufficient reaction between the new drug candidate material and the target material as injected to allow energy transfer including bioluminescence resonance energy transfer (BRET), fluorescence resonance energy transfer (FRET), or fluorescence polarization (FP) to occur, in which the reaction channel may be designed in a letter U configuration to ensure sufficient moving distance within a narrow space occupied by the microfluidic device. For example, the reaction may include immunological reaction such as antigen-antibody reaction (e.g., virus antigen-virus antibody, pathogenic microorganism-pathogen microorganism antibody, etc.), biotin-avidin, immunoglobulin G-protein A; hormone-hormon receptor reaction; DNA-DNA receptor reaction; RNA-RNA receptor reaction; or drug-drug receptor reaction, but not limited thereto.

Next, step 4 is a step injecting oil through the oil channel connected to the reaction channel of step 3 to form droplets.

To be specific, step 4 may injected the oil into a channel where the mixture of the new drug candidate material and the target material which have been reacted to each other flows, to thereby form reaction droplets of constant volume.

The oil may preferably have viscosity ranging from 5 to 20 cSt. If the oil has the viscosity less than 5 cSt, droplets may not form, and if the viscosity exceeds 100 cSt, the oil may not flow in the channel.

The oil may preferably include a silicon oil, mineral oil, or hexadecane, but not limited thereto.

The oil further may include a surfactant. By using the surfactant, it can be formed more stable droplets.

Wherein, the oil may be injected under pressure two to ten times greater than the pressure under which the mixture is injected. As the pressure of the injected oil is considered to be an important variable that plays an important role in the formation of the droplets, the size of the droplets may decrease as the pressure of injecting the oil is increased. If the injection pressure of the oil is less than the injection pressure of the mixture, the droplets may not form, while if the injection pressure of the oil exceeds the injection pressure of the mixture, the mixture may not be injected into the channel.

The droplets of step 4 may be sized to have 0.01 to 0.10 mm in length, and 1 nl to 100 pl in volume.

Next, step 5 is a step detecting the droplets exhibiting fluorescence or illumination from among the droplets formed at step 4, using the mass spectrometer. Using the mass spectrometer, it is possible to obtain mass-to-charge ratio of the reaction fluorescent material, define structure of a compound using high resolution spectrometer and also identify the structure of the new drug candidate material using multi-dimensional mass spectrometer.

Further, the present invention provides a microfluidic device, comprising: a new drug candidate material inlet to which a new drug candidate material is injected; a new drug candidate material channel through which the injected new drug candidate material is moved; a target material inlet to which a target material is injected; a target material channel through which the fed target material is moved; a reaction channel connected to the new drug candidate material channel and the target material channel so that reaction and moving of the new drug candidate material and the target material are carried out therein; an oil channel connected to the reaction channel, and comprising an oil inlet to which the oil is injected to form reaction droplets by blocking a flow of the new drug candidate material and the target material; and an outlet through which the reaction droplets and the oil are discharged.

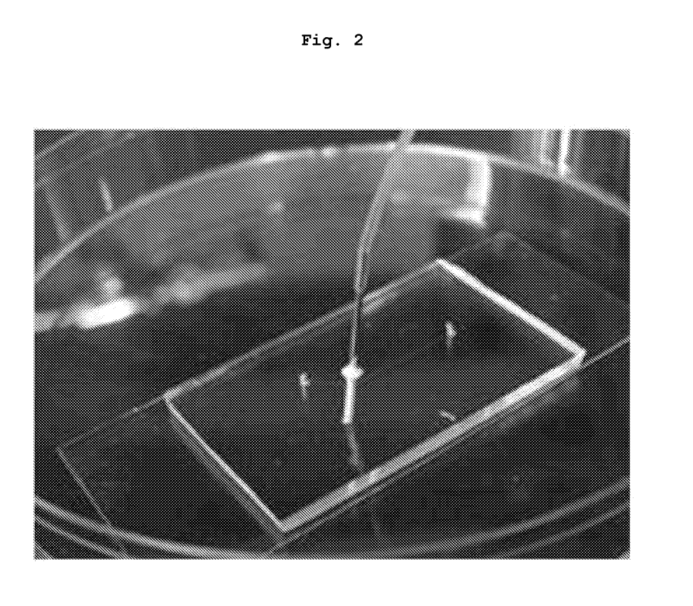

FIG. 2 depicts a microfluidic device according to an embodiment of the present invention.

Referring to FIG. 2, in the microfluidic device according to an embodiment of the present invention, the new drug candidate, separated from the liquid chromatograph, is injected into an inlet and moved along the new drug candidate material channel, the target material is injected through the inlet and moved along the target material channel, and the new drug candidate material and the target material are mixed and reacted with each other in the reaction channel which is connected to the respective channels.

The mixture of the new drug candidate material and the target material may form reaction droplets due to the oil injected thereto. The reaction droplets may move along the reaction channel, with exhibiting illumination or fluorescence which is detectable through a detector. The reaction droplets and the oil may be discharged through the outlet connected to an end of the reaction channel.

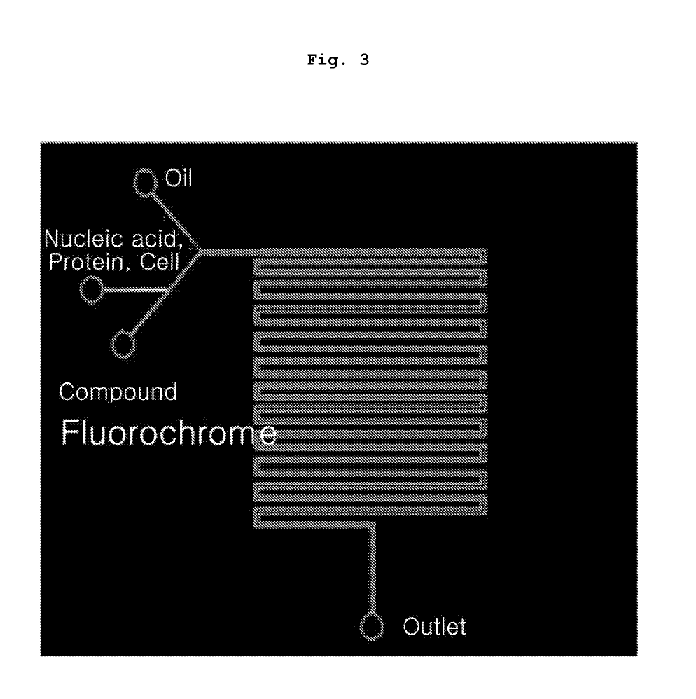

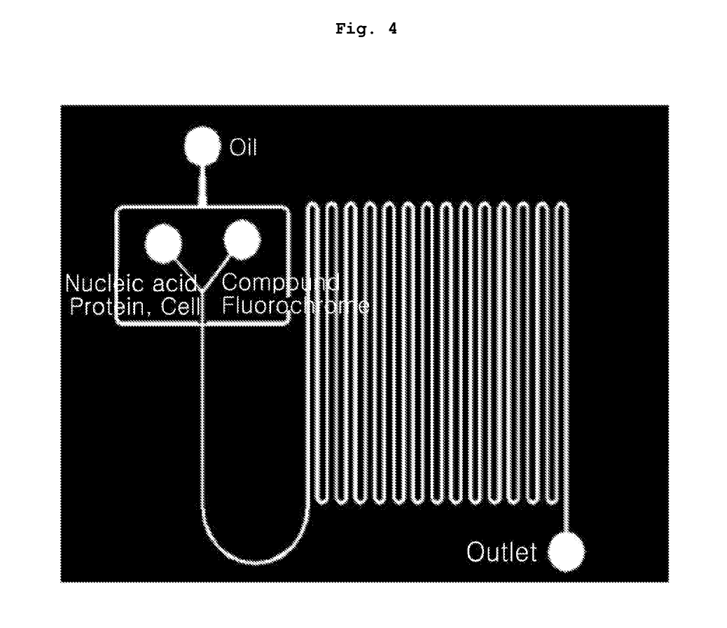

To ensure a sufficient moving distance of the mixture of the new drug candidate material and the target material inside a narrow space occupied by the microfluidic device, the reaction channel may preferably be formed into U-shape (see FIGS. 3 and 4).

The reaction channel according to the present invention may desirably be connected to the oil channel in T-, Y-, X- or +-shaped structure, but not strictly limited thereto.

In addition, the oil channel according to the present invention may include a neck designed to reduce the width of the oil channel by 5 to 20% at a connecting portion to the reaction channel. The neck reduces the width of the oil channel at a connecting portion to the reaction channel, to thereby partially increase the flow rate of the oil and still facilitate the formation of the reaction droplets.

The channel may preferably be 1 to 100 μm in width, and 1 to 100 μm in height. If the width is less than 10 μm, the pressure of the fluid in the channel may increase more than necessary, while if the width exceeds 100 μm, the size of the droplets may increase more than necessary.

The microfluidic device according to the present invention may be made from an optically transparent material to detect illumination and fluorescence expressed by a reaction between the new drug candidate material and the target material. Any material with optical transparency may be used as the optically transparent material without limit, which may include glass, quartz, polydimethylsiloxane (PDMS), polymethylmethacrylate, polyacrylate, or polycarbonate or polyurethane.

The microfluidic device may use glass as a substrate through surface and also use polymer as a material for the channel, and modificating and rinsing by chemical and physical methods, to thereby provide more reinforced bonding and also facilitated flow of the fluids along the channel.

Furthermore, the present invention provides a liquid chromatography/microfluidic device/mass spectrometry system, comprising: a liquid chromatograph which separates a new drug candidate material from a natural materials or synthesized new drug candidate materials; a splitter which is connected to the liquid chromatograph and splits into a microfluidic device and a mass spectrometer; the microfluidic device which allows the new drug candidate material separated from the splitter to react with a target substance; a detector which detects illumination or fluorescent reaction within the microfluidic device; and the mass spectrometer which analyzes the new drug candidate material separated from the splitter.

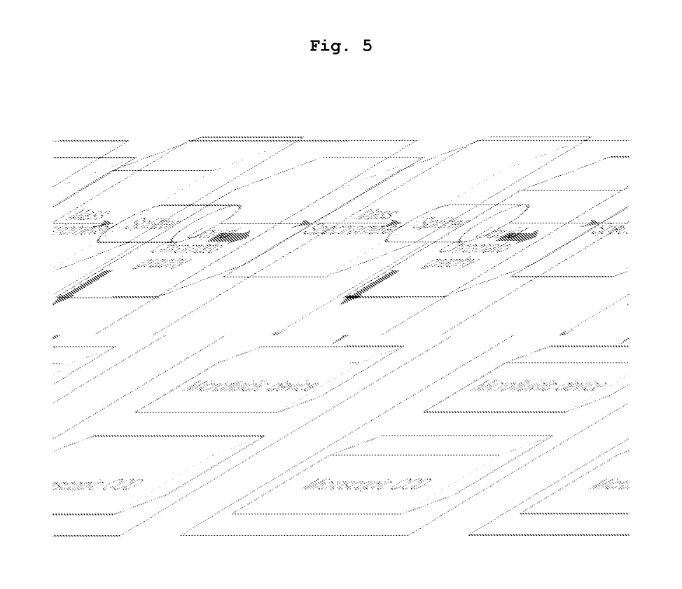

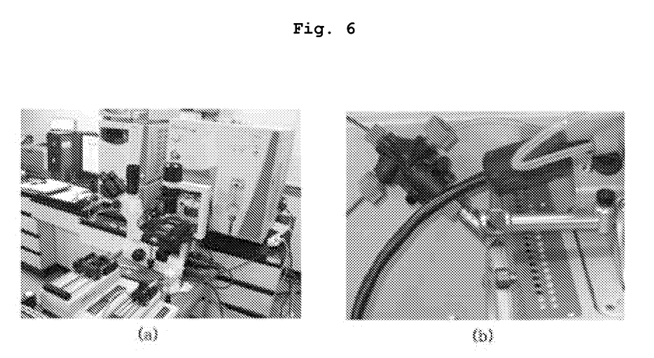

FIG. 5 illustrates a conceptual view of the liquid chromatography/microfluidic device/mass spectrometry system according to an embodiment of the present invention, and FIG. 6 illustrates the liquid chromatography/microfluidic device/mass spectrometry system according to an embodiment of the present invention as actually constructed.

Referring to FIG. 5, the splitter of the liquid chromatography/microfluidic device/mass spectrometry system according to an embodiment of the present invention may separately feed the new drug candidate separated from the liquid chromatograph into the microfluidic device and the mass spectrometer, respectively. The liquid chromatography/microfluidic device/mass spectrometry system constructed as explained above according to an embodiment of the present invention may concurrently feed the fraction separated from the liquid chromatograph into the microfluidic device and the mass spectrometer, detect illumination or fluorescent reaction within the microfluidic device with fluorescent microscopic probe, or the like, and find a substance as analyzed at the mass spectrometer to thereby screen the new drug candidate in a real-time basis.

The mass spectrometer may additionally include a nano-column at the inlet. The nano-column may reduce the flow rate fed into the mass spectrometer through the splitter to the same level as that of the flow fed into the microfluidic device. Accordingly, the nano-column operates to maintain the time at which the fraction of the liquid chromatograph is discharged, the time at which the fraction is analyzed at the mass spectrometer, and the time at which the fraction after reaction is detected within the microfluidic device.

Wherein, the nano-column may preferably reduce the flow rate of the flow fed from the splitter in a range of 1/350 and 1/250, and a shortcoming may occur if the flow rate diverges from the above range. That is, it may be difficult to detect the substance with accuracy due to difference between the time of analysis at the mass spectrometer and the time of detection.

Further the present invention provides a method for screening a new drug candidate using a liquid chromatography/microfluidic device/mass spectrometry system including: separating a natural material containing the new drug candidate material or a synthesized new drug material using a nano liquid chromatograph and injecting into the microfluidic device (step A); splitting the new drug candidate material injected into the microfluidic device at step A in the microfluidic device and injecting into a new drug candidate material channel (step B-1); injecting a target material into an inlet of a target material channel designed to be connected to a new drug candidate material channel for the new drug candidate material split at step B-1 (step B-2); injecting the new drug candidate material split at step B-1 into the mass spectrometer (step B-3); reacting the new drug candidate material injected at step B-1 with the target material injected at step B-2 in a reaction channel to which the channels of steps B-1 and B-2 are connected (step C); injecting an oil through an oil channel connected to the reaction channel of step C to form droplets (step D); and detecting the droplets in fluorescence or emitting light from among the droplets of step D, using the mass spectrometer (step E).

An embodiment of the present invention will be explained in greater detail below with reference to the respective steps thereof.

In one embodiment, the step A is a step separating a natural materials containing the new drug candidate materials or synthesized new drug materials using a nano liquid chromatograph and injecting into the microfluidic device.

At step A, the natural material containing the new drug candidate materials or the synthesized new drug materials is separated into a single new drug candidate material using chromatograph, and injected into the microfluidic device. The respective new drug candidates material may be separated at different time of discharge due to varying moving speed in columns. The chromatograph may desirably be a liquid chromatograph, or more preferably, nano-liquid chromatograph. The new drug candidates separated using chromatograph may be injected directly into the microfluidic device.

Next, the step B-1 is a step splittings the new drug candidate material injected into the microfluidic device and injecting the split material into the new drug candidate material channel.

The new drug candidate material injected into the microfluidic device may be injected into the new drug candidate material channel in the microfluidic device via a splitter provided in the microfluidic device.

Next, the step B-2 is a step injecting a target material into an inlet of a target material channel which is designed to be connected to the channel of the new drug candidate material split at step B-1. Step B-2 may be performed in the same manner as step 2-2.

Next, the step B-3 is a step injecting the new drug candidate material split at step B-1 into the mass spectrometer. It is preferable that step B-3 is performed concurrently with steps B-1, and B-2. Steps B-1, B-2 and B-3 may preferably be performed concurrently to provide appropriate reaction mixture to the reaction channel of step C.

Next, the step C is a step reacting the new drug candidate material injected at step B-1 with the target material injected at step B-2 in a reaction channel to which the channels of steps B-1 and B-2 are connected. Step C may preferably be performed in the same manner as in step 3.

Next, the step D is a step injecting an oil through an oil channel connected to the reaction channel of step C to form droplets. Step D may be performed in the same manner as step 4.

Next, the step E is a step detecting the droplets in fluorescence or emitting light from among the droplets of step D, using the mass spectrometer. Step E may be performed in the same manner as in step 5.

Furthermore, the present invention provides a microfluidic device including: a new drug candidate material inlet to which a new drug candidate material is injected from a nano liquid chromatograph; a splitter which splits the injected new drug candidate material into a mass spectrometer and a new drug substance material channel; a target material inlet to which a target material is injected; a target material channel through which the injected target material is moved; a reaction channel connected to the new drug candidate material channel and the target material channel so that reaction and moving of the new drug candidate material and the target material are carried out therein; an oil channel connected to the reaction channel, and comprising an oil inlet to which the oil is injected to form reaction droplets by blocking a flow of the new drug candidate material and the target material; and an outlet through which the reaction droplets and the oil are discharged.

In the microfluidic device according to an embodiment of the present invention, the new drug candidate material separated from the nano-liquid chromatograph is injected into the inlet, and the injected new drug candidate material is separated into the mass spectrometer and the new drug candidate channel inside the microfluidic device using the splitter. Wherein, the new drug candidate material moves to the new drug candidate material channel, the target material is injected into the inlet and moved to the target material channel, and the injected new drug candidate and the target substance are mixed and reacted with each other in the reaction channel to which the respective channels are connected. The mixture of the new drug candidate and the target substance may form reaction droplets due to oil as injected. The reaction droplets move along the reaction channel, during which illumination or fluorescence appears, and such can be detected via the detector. The reaction droplets and oil may be discharged through the outlet connected to an end of the reaction channel.

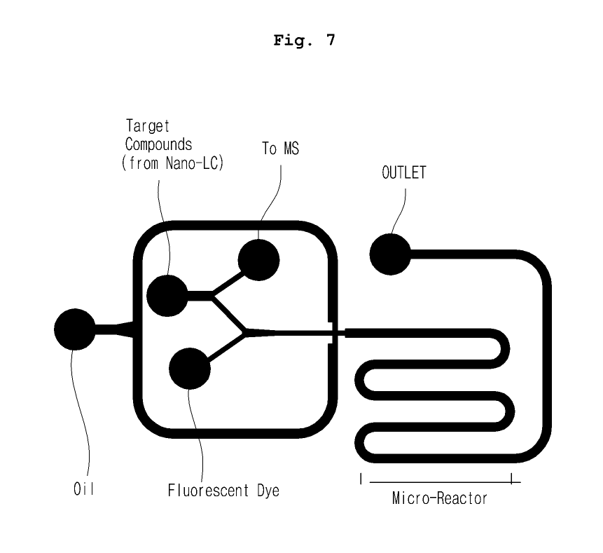

To ensure a sufficient moving distance of the mixture of the new drug candidate material and the target material inside a narrow space occupied by the microfluidic device, the reaction channel may preferably be formed into U-shape (see FIG. 7).

Wherein, the reaction channel according to the present invention may desirably be connected to the oil channel in a combination of two or more of T-, Y-, X- or +-shaped structures, but not strictly limited thereto.

Furthermore, the present invention provides a liquid chromatography/microfluidic device/mass spectrometry system including: a nano liquid chromatography which separates a new drug candidate material from natural materials or synthesized new drug candidate materials; a microfluidic device connected to the nano liquid chromatography; a splitter which separates an inside of the microfluidic device the mass spectrometer and a new drug candidate material channel; a detector which detects illumination or fluorescent reaction within the microfluidic device; and a mass spectrometer which analyzes the new drug candidate separated from the splitter.

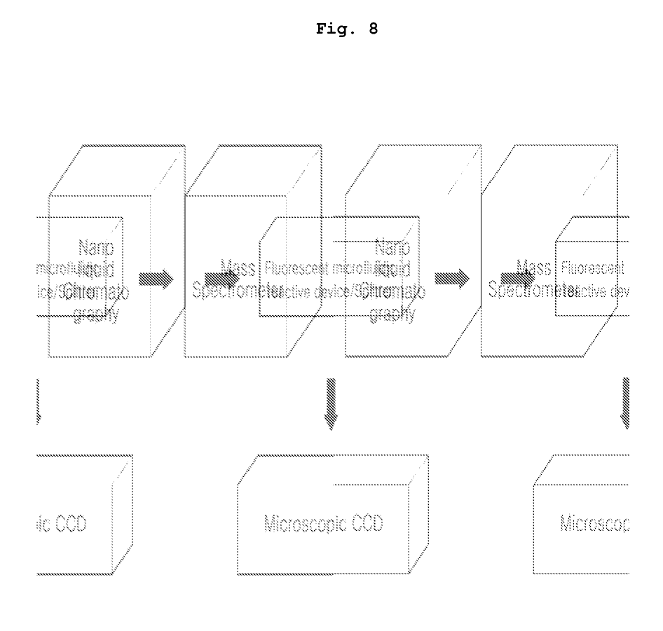

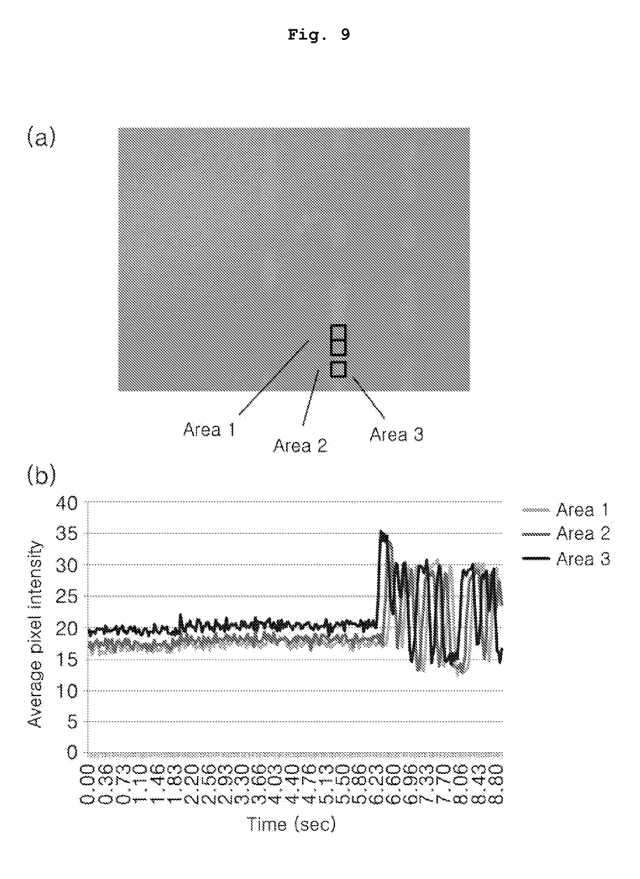

FIG. 8 illustrates a conceptual view of a liquid chromatography/microfluidic device/mass spectrometry system according to the present invention.

Referring to FIG. 8, the liquid chromatography/microfluidic device/mass spectrometry system according to the present invention injects a fraction separated from the nano-liquid chromatography into the microfluidic device, and separates the fraction in the microfluidic device into the mass spectrometer and the new drug candidate material channel in the microfluidic device using the splitter. The liquid chromatography/microfluidic device/mass spectrometry system constructed as above may detect illumination or fluorescence within the microfluidic device using microscopic fluorescence probe or the like, and at the same time, find a material analyzed at the mass spectrometer, to thereby screen the new drug substance in a real-time basis.

The mass spectrometer may additionally include a nano-column at the inlet. The nano-column may reduce the flow rate of the flow injected into the mass spectrometer through the splitter to the same level as that of the flow injected into the microfluidic device. Accordingly, the nano-column operates to maintain the time at which the fraction of the liquid chromatograph is discharged, the time at which the fraction is analyzed at the mass spectrometer, and the time at which the fraction after reaction is detected within the microfluidic device.

Where, the nano-column may preferably reduce the flow rate of the flow fed from the splitter in a range of 1/350 and 1/250, and a shortcoming may occur if the flow rate diverges from the above range. That is, it may be difficult to detect the substance with accuracy due to difference between the time of analysis at the mass spectrometer and the time of detection.

The liquid chromatography/microfluidic device/mass spectrometry system may have an integrated splitter within the microfluidic device, in which case the system may not require a separate splitter.

Furthermore, in one embodiment of the present invention, a splitter device may be provided, which separates nano-fluid in the microfluidic device and injects the nano-fluid into the mass spectrometer and the new drug candidate channel in the microfluidic device respectively.

In addition, the present invention provides a method for constructing a library in order to separate mixture of new drug candidate materials with liquid chromatography and to react the separated new drug material using the microfluidic device and illuminating phenomenon, when the new drug candidate material is discovered, and the method including: mixing mixture of pure compounds in a solvent to obtain a mixture solution (step 1); and identifying structures and information of the compounds contained in the mixture solution obtained at step 1 (step 2).

The method for constructing a library will be explained in greater detail below with reference to the respective steps.

The step 1 according to the present invention is a step mixing mixture of pure compounds in a solvent to obtain a mixture solution.

The compounds are reactable with the target material, and may preferably include synthesized compounds or natural materials. If the compounds are natural, natural extract is used for the library. It is preferable to prepare the mixture solution by mixing from 100 to 300 compounds to reduce the number of possibilities that the compounds may react with the target material. Further, it is preferable to use a compound with a known structure.

Next, the step 2 according to the present invention is a step is a step identifying structures and information of the compounds contained in the mixture solution obtained at step 1.

The library according to the present invention may be constructed by repeatedly performing steps 1 and 2. The library may be constructed by: performing re-separating with liquid chromatography; reacting with target material; and detecting compound reacted with the target material using mass spectrometer.

Furthermore, the present invention provides a method for identifying a reaction material when the new drug candidate material is reacted with the target material in the microfluidic device, by recording optically a reaction between a new drug candidate material and a target material and tracking a movement of the droplets and automatically detecting a change in brightness of the fluorescence in the droplets and tracking reaction time, in which the method including: filtering a fluorescence using a microscope and capturing a video image of the same using a CCD camera (step 1); stacking the video image having fluorescence progressed therein from among the video image acquired at step 1 into a single-layer image (step 2); setting a limited area within a fluid passage of a microfluidic device and measuring a change in luminous flux of pixels within the set area image by image (step 3); and exporting the luminous flux acquire at step 3 onto a spreadsheet to thereby detect a time at which the fluorescence appears (step 4).

Wherein, it is possible to detect the reaction material in which fluorescence appears, by matching the time at which the fluorescence appears at step 4 with the time of the mass spectrometer. The fluorescent image of step 3 and the spreadsheet of step 4 according to the identification method is showed in FIG. 9. More specifically, FIG. 9(a) illustrates an image which represents measurement of variation of luminous flux of the pixels at three regions (i.e., regions 1 to 3) in the fluorescent image where the fluorescence is progressed, and FIG. 9(b) represents variations of luminous flux at regions 1 to 3 compared with respect to time divisions, in which the time detected as having appearance of fluorescence is identified. Referring to FIG. 9(b), region 1 has fluorescence generated at about 6.62 sec, region 2 at about 6.33 sec, and region 3 about at 6.40 sec The above result indicates that the material detected at about 6.62 second by the mass spectrometer corresponds to the reaction material of region 1, the material detected at about 6.53 sec corresponds to the reaction material of region 2, and the material detected at about 6.40 sec corresponds to the reaction material of region 3.

Further, the present invention provides an apparatus for identifying a reaction material when the new drug candidate material is reacted with the target material in the microfluidic device, by recording optically a reaction between a new drug candidate material and a target material and tracking a movement of the droplets and automatically detecting a change in brightness of the fluorescence in the droplets and tracking reaction time, in which the apparatus including:

a microscopic fluorescent probe which filters a fluorescence; a CCD camera which captures the fluorescence into a video image; an image program which stacks the video image into a single-layer image; and a program which measures a change in luminous flux of pixels.

### Mode for Invention

Hereinbelow, the present invention will be explained in greater detail with reference to exemplary embodiments and the accompanying drawings. It should be noted that the exemplary embodiments are written only for illustrative purpose, and therefore, should not be construed as limiting the present invention.

### EXAMPLE 1

### Fabrication of Microfluidic Device

A microfluidic device was fabricated using negative photolithography which will be explained below.

Photoresist was evenly applied on a silicon wafer under vacuum using spin coater, and activated by the light while using a photomask image. A master was fabricated by leaving only the channels. The channels were formed into a pattern according to which the channel for feeding a mixture and a channel for feeding oil form a Y-shaped structure.

Trimethylcholrosilane (TMCS) was coated on the prepared master to prevent binding between the silicon wafer and PDMS, on which the mixture of polydimethylsiloxane (PDMS, Sylgard 184) oligomer and cross-linker in 4:1 ratio was poured. The above was hardened in the oven for 30 minutes at 75° C., a portion with the channels is removed, and connecting holes were bored so that solution can be inserted into the channel therethrough.

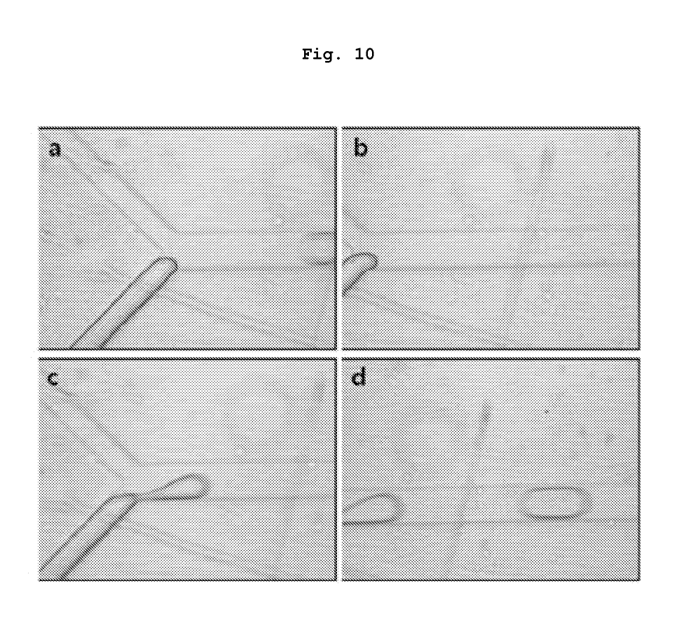

Solution, in which PDMS oligomer and hardner were mixed in 20:1 ratio, was spin-coated on a glass substrate. After hardening in the oven for 30 minutes, the PDMS with the channels prepared in advance was adhered to the lower substrate, and underwent hardening for additional 30 minutes in the oven. As a result, PDMS microfluidic reaction device was fabricated. A connecting line, through which solution is fed, is connected to the fabricated chip as illustrated in FIG. 2. The microfluidic device fabricated in the manner explained above was illustrated in FIG. 10.

Silicon oil (viscosity: 10 cSt, KF-96, ShinEtsu) was injected into the microfluidic device at a flow rate of 10 μl/hour, and aqueous solution containing blue pigment was injected at a flow rate of 15 μl/hour into the microfluidic device fabricated in the manner explained above, so that the pressure of the oil and the mixture reached an equilibrium value after which it was confirmed that the uniform droplets as illustrated in FIG. 10 are continuously generated.

### EXAMPLE 2

### Fabrication of Microfluidic Device 2

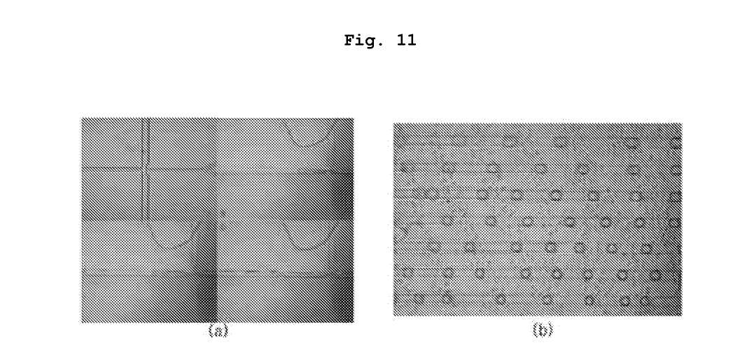

The microfluidic device was fabricated in the same manner as used in Example 1, except that a channel pattern is formed so that the channel for feeding a mixture and a channel for feeding oil form a T-shaped structure. Then, aqueous solution containing blue pigment was injected into the microfluidic device fabricated in the manner explained above, under 1 psig, and at a flow rate of 10 μl/hour. Mineral oil (21 cSt) with viscosity approximately two times greater than that of silicon oil, was injected at a flow rate of 15 μl/hour to form droplets. As illustrated in FIG. 11(a), droplets were formed. FIG. 11(b) illustrates uniform-sized droplets formed.

### EXAMPLE 3

### Fabrication of Microfluidic Device 3

The PDMS surface was treated with O₂ plasma for 20 seconds to directly bond the PDMS with the channel pattern fabricated in Example 1 to the glass substrate, then it was joined with the glass substrate was followed by bonding in the oven for 12 hours. Wherein, the glass substrate was treated with a mixture of sulfur and hydrogen peroxide or plasma treated so that organic matter was removed from the surface and the microfluidic device was bonded more firmly when completed.

### EXAMPLE 4

### Fabrication of Microfluidic Device 4

The microfluidic device was fabricated in the same manner as explained above in Example 1 or Example 2, except that surfactant SPAN 80 was additionally mixed with oil and used.

### EXAMPLE 5

### Fabrication of Microfluidic Device with Integrated Splitter 1

The microfluidic device was fabricated using negative photolithography which will be explained below.

Photoresist was evenly applied under vacuum on a silicon wafer using spin coater, and activated by the light while using a photomask image. A master was fabricated by leaving only the channels. The channels were formed in such a pattern that a channel for feeding a mixture and a channel for feeding oil are connected with each other in a Y-shaped structure. Additionally, a Y-shaped channel pattern is formed within the microfluidic device to thereby integrate the Y-shaped splitter which separates the nano fluid fed from the nanofluidic chromatograph into feeds into the mass spectrometer and the mixture feeding channel within the microfluidic device. In Examples 1 to 3 explained above, the flow rate of the fluid fed from the liquid chromatograph into the microfluidic device is regulated by the valve. In Example 4, the valve may be omitted by integrating the splitter within the microfluidic device, since the nano fluid fed from the liquid chromatograph can be directly separated and fed without requiring use of a valve.

Trimethylcholrosilane (TMCS) was coated on the prepared master to prevent binding between the silicon wafer and PDMS, on which the mixture of polydimethylsiloxane (PDMS, Sylgard 184) oligomer and cross-linker in 4:1 ratio was poured. The above was hardened in the oven for 30 minutes at 75° C., a portion with the channels is removed, and connecting holes were bored so that solution can be inserted into the channel therethrough.

Solution, in which PDMS oligomer and hardner were mixed in 20:1 ratio, was spin-coated on a glass substrate. After hardening in the oven for 30 minutes, the PDMS with the channels prepared in advance was adhered to the lower substrate, and underwent hardening for additional 30 minutes in the oven. As a result, PDMS microfluidic reaction device was fabricated.

### EXAMPLE 6

### Fabrication of Microfluidic Device with Integrated Splitter 2

Except for the fact that a T-shaped channel pattern was formed to connect the mixture feeding channel and the oil feeding channel, the microfluidic device was fabricated in the same manner as explained above with reference to Example 5.

### EXAMPLE 7

### Fabrication of Microfluidic Device with Integrated Splitter 3

In order to directly bond the PDMS in which the channel is formed according to Example 5, to a glass substrate, the PDMS surface was treated with O₂ plasma for 20 seconds, joined to the glass substrate and bonded in an oven for 12 hours. By treating the glass substrate with a mixture of sulfur and hydrogen peroxide, or plasma to remove organic substance from the surface, a more firmly bonded microfluidic device was fabricated.

### EXAMPLE 8

### Fabrication of Microfluidic Device with Integrated Splitter 4

Except that a surfactant was mixed with the oil, a microfluidic device was fabricated in the same manner as that of Example 5 or 6.

### EXPERIMENTAL EXAMPLE 1

### Detection of Fluorescence of Droplets in Microchannel

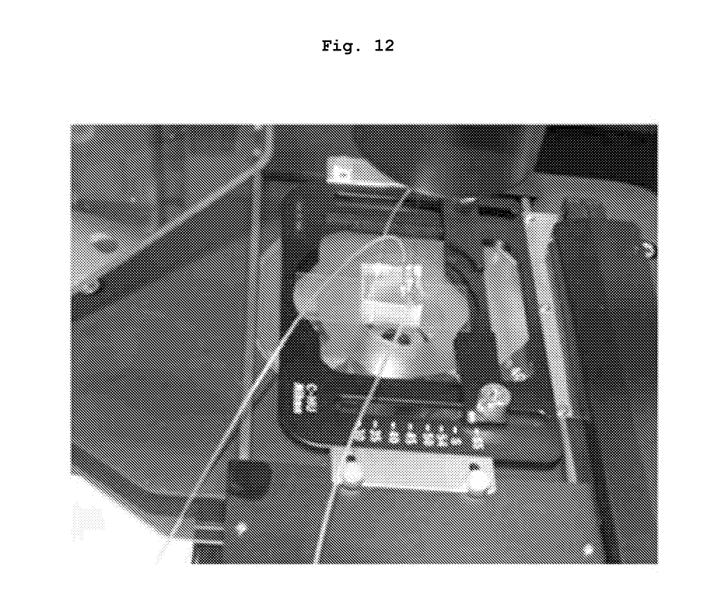

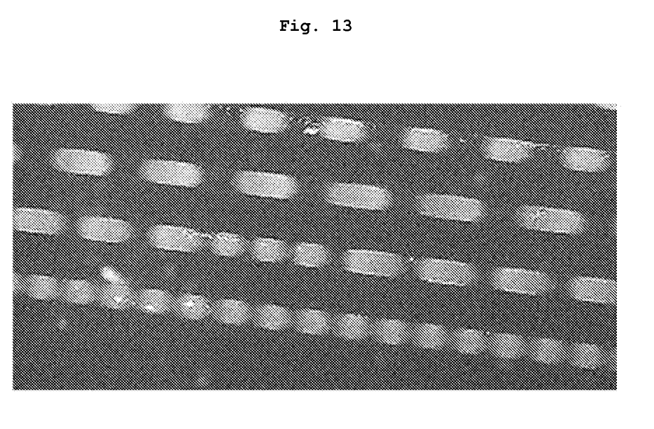

A mixture of DAPI(4′6-diamidino-2-phenylindole, excitation wavelength of 345 nm, emission wavelength of 458 nm) and acrydine orange (excitation wavelength of 500 nm, emission wavelength of 530 nm) was injected to a new drug candidate material inlet of a microfluidic device in a flow rate of 10 to 15 μl/hour, and herring DNA in concentration of 8 mg/ml was injected into a target substance inlet in a flow rate of 10 to 15 μl/hour. At this time, inside of the reaction channel was maintained to be under 1 psig of pressure, and the oil was injected under 2.5 psig of pressure to form droplets. The formed droplets were detected using fluorescent microscope (Nikon inverted microscope, Eclipse Nikon TS100, 4×10 UV lens, 100 W Nikon Intensilight epi-fluoresence lamp). FIG. 12 shows the microfluidic device mounted on the fluorescent microscope, and FIG. 13 illustrates the result of detecting fluorescence using the above.

Referring to FIG. 13, the droplets formed according to the present invention fluoresced and were formed uniformly.

### EXPERIMENTAL EXAMPLE 2

### Detection of Mixture using Microfluidic Device

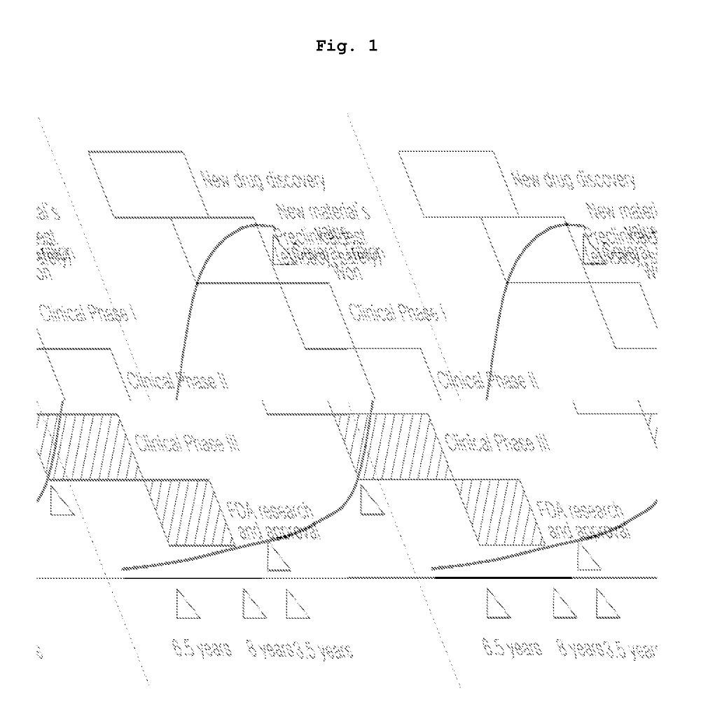

Herring DNA in concentration of 8 mg/ml was fed in a flow rate of 10 to 15 μl/hour into the target material inlet of the screening device constructed as explained above with reference to FIGS. 5 and 6 using the microfluidic device as shown in FIG. 1, 2 or 4, and a mixture of DPI and acrydine orange was separated with a liquid chromatography (Finnigan Surveyor LC), and the separated fraction was injected into the splitter. The fraction injected into the splitter was reduced in its flow rate to 1/300 through the nano column (Polymicro OD 359/ID250/tube coat 17.5) and then injected into the new drug candidate material inlet and the mass spectrometer (Finnigan TSQ MS Quantum Ultrainnigan TSQ MS Quantum Ultra).

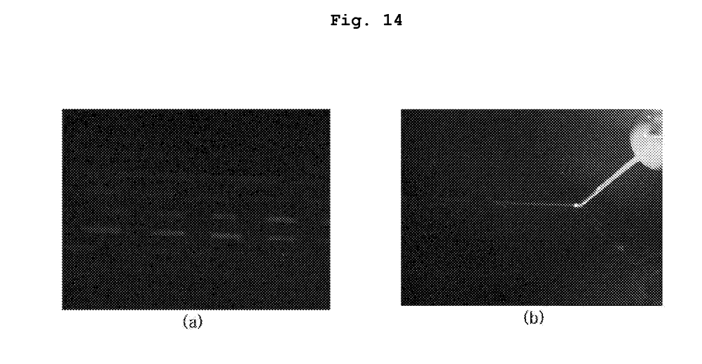

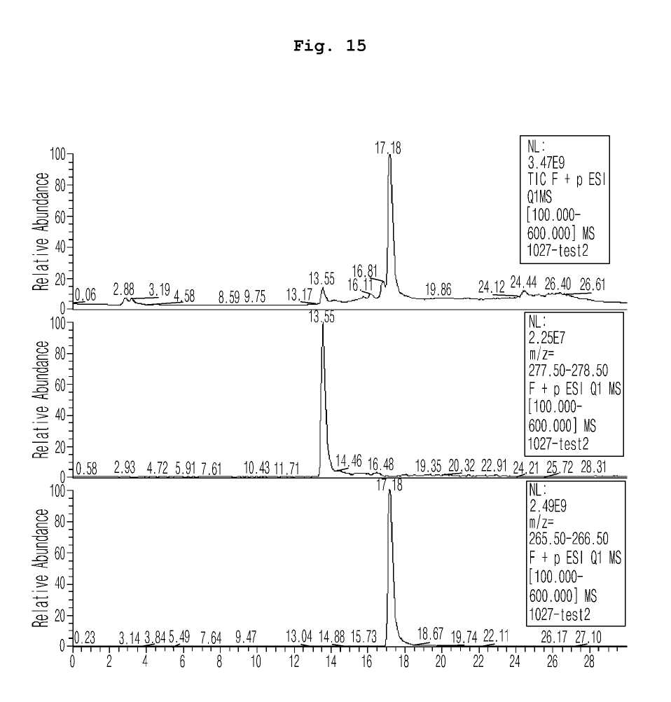

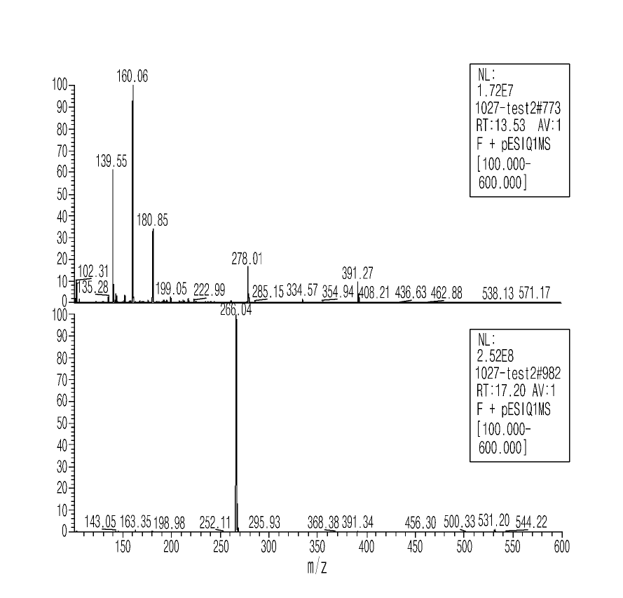

FIG. 14 illustrates the process of observing the microfluidic device using the microscopic fluorescence probe, and FIG. 15 illustrates the Total Ion Chromatography (TIC) obtained as a result of screening in the manner explained above.

Referring to FIG. 14, the fraction fed into the microfluidic device reacted with the target material (i.e., Herring DNA) to fluoresce at the fluorescence probe.

Further, referring to FIG. 15, the mixture of DAPI and acrydine orange was separated trough the liquid chromatography into DAPI at 12.55 minute and acrydine orange at 17.18 minute, and the mass spectrometer confirmed that the separated fractions were DPI and acrydine oranges, respectively, thereby indicating that the new drug candidate material can be screened in real-time basis as these are separated at the liquid chromatography.

### EXPERIMENTAL EXAMPLE 3

### Detection of Protein Fluorescence of Droplets in Microchannel with Integrated Splitter

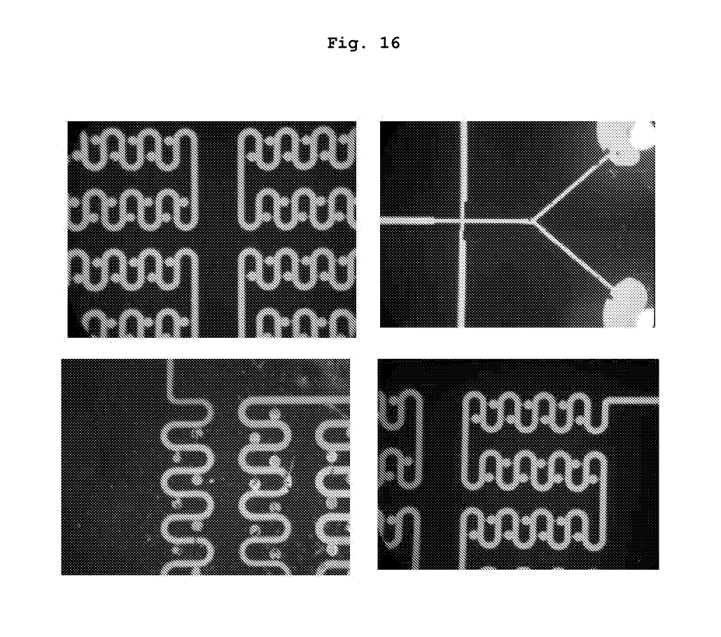

Dronpa (emission wavelength 520 nm) in concentration approximately of 1 mg/ml was injected into a new drug candidate material inlet of the microfluidic device of Examples 5 to 8 in a flow rate of 10 to 15 μl/hour. The inside of the reaction channel was maintained under 1 psig of pressure, and the oil was fed under 2.5 psig of pressure to form droplets. The formed droplets were detected using fluorescent microscope (Nikon inverted microscope, Eclipse Nikon TS100, 4×10 UV lens, 100 W Nikon Intensilight epifluorescence lamp), and FIG. 16 illustrates the result of the detection.

Referring to FIG. 16, the droplets formed according to the present invention fluoresced and were formed uniformly.

1. A method for screening a new drug candidate material using a liquid chromatography/microfluidic device/mass spectrometry system, the method comprising:
separating a natural material containing the new drug candidate material or a synthesized new drug material using a liquid chromatograph (step 1);
injecting the new drug candidate material separated at step 1 into inlets of a mass spectrometer and of a microfluidic device at the same time through a splitter (step 2-1);
injecting a target material into an inlet of a target material channel designed to be connected to a new drug candidate material channel for the new drug candidate material separated at step 1 (step 2-2);
reacting the new drug candidate material and the target material injected at steps 2-1 and 2-2 respectively in a reaction channel connected with the channels of steps 2-1 and 2-2 (step 3);
injecting an oil through an oil channel connected to the reaction channel of step 3 to form droplets (step 4); and detecting the droplets in fluorescence or emitting light from among the droplets of step 4, using the mass spectrometer (step 5).

2. The method according to the claim 1, wherein the new drug candidate material of step 2-1 or the target material of step 2-2 are injected in 1 to 20 μl/hour.

3. The method according to the claim 1, wherein the target material of step 2-2 is a disease-associated substance selected from a group consisting of DNA, protein and cell.

4. The method according to the claim 3, wherein the target material is a material which can induce or increase fluorescence or illumination by reacting with the new drug material.

5. The method according to the claim 1, wherein processes at steps 2-1 and 2-2 are carried out at the same time.

6. The method according to the claim 1, wherein the target material of step 2-2 is in concentration of 0.1 to 10 mg/ml.

7. The method according to the claim 1, wherein the oil of step 4 is non-polar, and has viscosity ranging from about 5 to about 20 cSt.

8. The method according to the claim 1, wherein the oil of step 4 is one selected from a group consisting of silicon oil, mineral oil, and hexadecane, or a mixture thereof

9. The method according to the claim 1, wherein the oil of step 4 further include a surfactant.

10. The method according to the claim 1, wherein the oil of step 4 is injected under 2 to 10 times greater pressure than the pressure of a mixture of the new drug candidate material and the target material.

11. A microfluidic device, comprising:
a new drug candidate material inlet to which a new drug candidate material is injected;
a new drug candidate material channel through which the injected new drug candidate material is moved;
a target material inlet to which a target material is fed; a target material channel through which the fed target material is moved;
a reaction channel connected to the new drug candidate material channel and the target material channel so that reaction and moving of the new drug candidate and the target substance are carried out therein;
an oil channel connected to the reaction channel, and comprising an oil inlet to which the oil is injected to form reaction droplets by blocking a flow of the new drug candidate material and the target material; and
an outlet through which the reaction droplets and the oil are discharged.

12. The microfluidic device according to the claim 11, wherein the reaction channel has a densely-repeating U-shaped channel structure to ensure a length of the channel.

13. The microfluidic device according to the claim 11, wherein the reaction channel and the oil channel are connected to each other in a T, Y, X or +-shaped structure.

14. The microfluidic device according to the claim 11, wherein the oil channel comprises a neck designed at a linking portion with the reaction channel to reduce a width of the oil channel by 5 to 20%.

15. The microfluidic device according to the claim 11, wherein the microfluidic device is formed from an optically-transparent material.

16. The microfluidic device according to the claim 15, wherein the optically-transparent material is one selected from a group consisting of glass, quartz, polydimethylsiloxane (PDMA), polymethylmethacrylate, polyacrylate, polycarbonate, and polyurethane, or a mixture thereof.

17. A liquid chromatography/microfluidic device/mass spectrometry system, comprising:
a liquid chromatograph which separates a new drug candidate material from natural materials or synthesized new drug candidate materials;
a splitter which is connected to the liquid chromatograph and splits into a microfluidic device and a mass spectrometer;
a microfluidic device which allows the new drug candidate material separated from the splitter to react with a target substance;
a detector which detects illumination or fluorescent reaction within the microfluidic device; and
a mass spectrometer which analyzes the new drug candidate material separated from the splitter.

18. The system according to the claim 17, wherein the mass spectrometer further comprises a nano column at an inlet thereof.

19. The system according to the claim 18, wherein the nano column reduces a flow rate of the new drug candidate fed via the splitter to 1/350 to 1/250.

20-43. (canceled)
